(19) United States
(12) Patent Application Publication
(10) Pub. No.: US 2013/0040839 A1
(43) Pub. Date: Feb. 14, 2013

(54) Method for Diagnosing or Determining the Prognosis of Colorectal Cancer (CRC) Using Novel Autoantigens: Gene Expression Guided Autoantigen Discovery

(71) Applicants: Mark J. Lim, Reading, MA (US); Christopher Sears, Watertown, MA (US); Kenneth J. Rothschild, Newton, MA (US)

(72) Inventors: Mark J. Lim, Reading, MA (US); Christopher Sears, Watertown, MA (US); Kenneth J. Rothschild, Newton, MA (US)

(73) Assignee: AMBERGEN, INC.

(21) Appl. No.: 13/532,025

(22) Filed: Jun. 25, 2012

Related U.S. Application Data
(60) Provisional application No. 61/501,466, filed on Jun. 27, 2011.

(51) Int. Cl. C40B 30/04; G01N 33/574

(52) U.S. Cl. 506/9; 435/7.92; 435/7.4

(57) ABSTRACT

The invention relates to the discovery and use of novel antigens/autoantigens, polyclonal and monoclonal antibodies/autoantibodies thereto, and in particular methods of using the antigens/autoantigens and antibodies/autoantibodies in the diagnostic, prognostic, staging and therapeutic regimens for the control of colorectal cancer.

### CROSS-REFERENCE TO RELATED APPLICATIONS

The present application claims the benefit of U.S. Provisional Patent Application No. 61/501,466, filed on. Jun. 27, 2011, which is incorporated herein by reference.

### FIELD OF THE INVENTION

This invention relates to molecular and protein biology, biochemistry, cell biology, immunology, immune response profiling, immunoassays, medicine and medical diagnostics. More specifically, the invention relates to novel antigens/autoantigens, polyclonal and monoclonal antibodies/autoantibodies thereto, and methods of using the antigens/autoantigens and antibodies/autoantibodies in the diagnostic, prognostic, staging and therapeutic regimens for the control of colorectal cancer. Furthermore, the invention relates to novel methods for discovery of novel antigens/autoantigens, polyclonal and monoclonal antibodies/autoantibodies thereto, said antigens/autoantigens and antibodies/autoantibodies used in the diagnostic, prognostic, staging and therapeutic regimens for the control of cancers and autoimmune diseases.

### BACKGROUND OF THE INVENTION

With almost 150,000 new cases each year, resulting in approximately 50,000 deaths, colorectal cancer (CRC) is the second most diagnosed cancer and the secondleading cause of cancer-related mortalities in the US [Ries LAG, Melbert D et al. (1975-2005)]. Although risk levels can vary based on gender and race, both males and females of all races and socioeconomic status are susceptible and need to be screened equally [Jackson-Thompson, Ahmed, German, Lai and Friedman (2006) Cancer 107: 1103-11]. In the entire population as a whole, the lifetime risk for developing CRC in the US as of 2005 was estimated to be 5.29% [Ries LAG, Melbert D et al. (1975-2005)]. Based on the current population of 305 million reported by the US Census Bureau, 18 million people currently living in US will develop CRC at some point during their lifetime.

Treatment of CRC is most effective when the disease is diagnosed early, while the cancer is still localized. In comparing the 5-year survival rates at various stages of the disease, the ability to treat CRC is reduced drastically from 90% or better when diagnosed early, to 68% at best when diagnosis occurs after the cancer infiltrates into deeper tissue layers and/or begins metastasis to other organs [Ries LAG, Melbert D et al. (1975-2005)]. With these statistics in mind, it is estimated that almost two-thirds of CRC-related deaths, or approximately 35,000 lives yearly, could currently be prevented with proper screening of the entire recommended population [Jackson-Thompson, Ahmed et al. (2006) Cancer 107: 1103-11]. Unfortunately, recent data indicate that only 34% of the recommended population is currently being screened [Subramanian, Klosterman, Amonkar and Hunt (2004) Prev Med 38: 536-50; Vijan, Inadomi, Hayward, Hofer and Fendrick (2004) Aliment Pharmacol Ther 20: 507-15], resulting in only 37% of CRC cases currently being caught early, when treatment is most effective [Ries LAG, Melbert D et al. (1975-2005)].

As the overall number of yearly CRC-related deaths has been decreasing only slightly, even with the many recent advances in cancer treatments [Xu, Zhou, Fung and Li (2006) Histol Histopathol 21: 867-72], it is very clear that the largest hurdle preventing greater success in treating CRC is the lack of proper screening and early detection [Jackson-Thompson, Ahmed et al. (2006) Cancer 107: 1103-11]. One of the most glaring road blocks in CRC screening is the extreme disparity between the demand and capacity for the most effective and highly recommended method, the colonoscopy [Vijan, Inadomi et al. (2004) Aliment Pharmacol Ther 20: 507-15]. The majority of new CRC cases, approximately 75%, are sporadic and occur in individuals with a median age of diagnosis of 71. With these statistics in mind, the American College of Gastroenterologist's most recent guidelines suggest that average risk individuals begin screening by colonoscopy once every ten years at age 50 [Rex, Johnson, Anderson, Schoenfeld, Burke and Inadomi (2009) Am J Gastroenterol 104: 739-50]. Based on most recent population data, this would account for 30% of the US population, or approximately 92 million people. Additionally, there is a strong genetic component to the disease, yielding a group at higher risk for developing CRC and accounting for the remaining 25% of all cases. Individuals in this group, who are susceptible to developing CRC as early as their twenties, include those with family histories and with known genetic predispositions such as familial adenomatous polyposis (FAP) and hereditary nonpolyposis colorectal cancer (HNPCC) [Lynch and de la Chapelle (2003) N Engl J Med 348: 919-32]. This group encompasses a significantly sized subset of the population under 50 who would normally not fall under the recommended guidelines for CRC screening. Based on most stringent guidelines, up to 3% of the population, an additional 9 million people, should be considered high risk due to family history and would benefit from colonoscopy screening beginning prior to the age of 50, with screens being repeated at more frequent intervals [Mitchell, Campbell, Farrington, Brewster, Porteous and Dunlop (2005) Br J Surg 92: 1161-4]. With over one third of the US population recommended for CRC screening at least once every 10 years, many requiring more frequent screening, sometimes as often as every 1-2 years, it is not surprising that the capacity is not high enough to meet these demands.

As of 2004, the estimated annual demand for colonoscopies to screen the entire recommended US population was around 8 million [Vijan, Inadomi et al. (2004) Aliment Pharmacol Ther 20: 507-15]. In some areas, this demand was over two times the capacity [Butterly, Olenec, Goodrich, Carney and Dietrich (2007) Am J Prev Med 32: 25-31]. Calculations performed in 2004 indicated that to meet these demands required for a significant reduction in the number of annual CRC-related deaths, an estimated 32,000 new gastroenterologists would be required. Even were this number attainable, costs for training new endoscopists, setting up new facilities, and additional yearly salaries could be prohibitive [Vijan, Inadomi et al. (2004) Aliment Pharmacol Ther 20: 507-15]. Additional obstacles, such as high cost to the uninsured and fear of procedure itself also dramatically reduce the screening rate [Subramanian, Klosterman et al. (2004) Prev Med 38: 536-50]. Together, this data emphasizes the need for a cheaper, non-invasive, high-throughput method for effective, early detection of CRC.

The alternative to the colonoscopy currently recommended by the American College of Gastroenterology, is an improved fecal occult blood test (FOBT), the fecal immunochemical test (FIT) [Rex, Johnson et al. (2009) Am J Gastroenterol 104: 739-50]. This test is more affordable and has a higher capacity to screen the recommended population, but has many pitfalls including low sensitivity (5.4%-62.6%), especially in detecting early stage adenomas (32%), and the requirement for actions many patients find unpleasant and are hesitant to perform such as a restriction in diet and specific stool collection procedures [Burch, Soares-Weiser, St John, Duffy, Smith, Kleijnen and Westwood (2007) J Med Screen 14: 132-7]. Recently, there has been a major shift in direction to try and develop alternative assays for CRC detection based on the identification of biomarkers, mainly nucleic acid-based, in blood and stool. These include assays for DNA methylation and mutation detection, as well as for detection of microRNAs [Kann, Han, Ahlquist, Levin, Rex, Whitney, Markowitz and Shuber (2006) Clin Chem 52: 2299-302; Kent Moore, Smith, Whitney, Durkee and Shuber (2008) Biotechniques 44: 363-74; Brenner, Benjamin et al. (2009) ASCO Gastrointestinal Cancers Symposium]. As of yet, none of these potential assays have made it to the clinic. One potential reason is the difficulties associated with isolation and detection of nucleic acids from blood and stool due to their very low concentrations and instability, indicating a market for non-nucleic acid-based assays. Together, the expanse of resources and efforts being directed towards developing new diagnostics for CRC further emphasizes the flaws of the current screening methods.

Another type of biomarker-based assay for cancer detection that is rapidly gaining more promise is the identification of proteins specifically expressed, or altered, in cancer cells called tumor associated antigens/autoantigens (TAAs) [Casiano, Mediavilla-Varela and Tan (2006) Mol Cell Proteomics 5: 1745-59; Belousov, Kuprash, Sazykin, Khlgatian, Penkov, Shebzukhov and Nedospasov (2008) Biochemistry (Mosc) 73: 562-72]. Several TAAs for CRC have been reported in the literature, and evidence suggests that autoantibodies against some of these TAAs are present in patient sera. In one study, the use of SEREX (serological identification of antigens by recombinant expression cloning) resulted in the identification of 8 different potential clones for TAAs, three of which (C210RF2, EPRS and NAP1L1) were found mainly in colorectal cancer patients' sera [Line, Slucka, Stengrevics, Silina, Li and Rees (2002) Cancer Immunol Immunother 51: 574-82]. WT1, which has been shown to be overexpressed, stimulates cytotoxic T-cells making it a candidate for anti-CRC-vaccine development [Koesters, Linnebacher, Coy, Germann, Schwitalle, Findeisen and von Knebel Doeberitz (2004) Int J Cancer 109: 385-92]. Other TAAs associated with CRC include colorectal tumor-associated antigen-1 (COA-1) [Maccalli, Li, El-Gamil, Rosenberg and Robbins (2003) Cancer Res 63: 6735-43], tumor-associated antigen 90K/Mac-2-binding protein [Ulmer, Keeler, Loh, Chibbar, Torlakovic, Andre, Gabius and Laferte (2006) J Cell Biochem 98: 1351-66] and tumor-associated antigen TLP [Guadagni, Graziano, Roselli, Mariotti, Bernard, Sinibaldi-Vallebona, Rasi and Garaci (1999) Am J Pathol 154: 993-9].

Autoantibody biomarkers against TAAs have several advantages over nucleic acid biomarkers including stability and “the inherent amplification of signals provided by the host's own immune system to low levels of tumor-associated antigens in early disease” [Storr, Chakrabarti, Barnes, Murray, Chapman and Robertson (2006) Expert Rev Anticancer Ther 6: 1215-23]. Although autoantibodies have been identified against some CRC-specific TAAs, for several of these antigens the presence of an autoantibody response is yet to be determined. For many of those autoantibodies that have been identified, several different assays were used and sufficient care was not taken in choosing sample sizes and collecting/reporting details that critically impact the strength of the data and its interpretation such as sample annotation (CRC stage, treatments prior to collection, etc). As a result, reported frequencies of autoantibodies against the same antigen, such as p53, in CRC patients often vary significantly [Scanlan, Chen et al. (1998) Int J Cancer 76: 652-8; Saleh, Kreissler-Haag and Montenarh (2004) Int J Oncol 25: 1149-55; Nozoe, Yasuda, Honda, Inutsuka and Korenaga (2007) Hepatogastroenterology 54: 1422-5]. Overall, the frequency at which individual autoantibodies present in cancer patients tends to be low, around 15-20% [Casiano [Casiano, Mediavilla-Varela et al. (2006) Mol Cell Proteomics 5: 1745-59; Belousov, Kuprash et al. (2008) Biochemistry (Mosc) 73: 562-72]. Thus a sensitive, high throughput method to screen large sample numbers and meticulously validate the presence and frequency of autoantibodies in CRC patient sera is urgently needed.

Tumorigenesis occurs as several cellular pathways become deregulated, or aberrant, due to changes in expression levels or mutation of cellular proteins, or TAAs. As many cancers tend to elicit a humoral immune response against these TAAs [Casiano, Mediavilla-Varela et al. (2006) Mol Cell Proteomics 5: 1745-59; Belousov, Kuprash et al. (2008) Biochemistry (Mosc) 73: 562-72], one can potentially use autoantibody profiling as a mechanism of understanding the biology of cancer cells. Additionally, analysis of potential changes in autoantibody panels as the disease progresses could yield important information on mechanisms regulating this progression. For example, many proteins that are required for migration and invasion are overexpressed at later stages and may elicit autoantibody responses specific for these stages, but not in patients with early stage CRC. Although such biomarkers would be less valuable as a diagnostic tool, they could serve as very useful targets for novel therapies targeting later stage CRC, for which effective treatments are currently lacking and the 5 year survival rate is relatively low [Ries LAG, Melbert D et al. (1975-2005)]. For example, one of the more recent, exciting, and promising focuses of current research on treating CRC is the development and use of new biologics directly targeting deregulated molecular pathways in cancer cells [Cohen and Hochster (2008) Gastrointest Cancer Res 2: 145-51].

This highlights the need for the discovery and validation of additional TAA biomarkers to be used in solid-phase immunoassays for the optimal diagnosis of cancers such as CRC. The most effective methods for the discovery of biomarkers such as TAAs are proteomics-based. Proteomics can be defined as the global (e.g. parallel or simultaneous) analysis of the entire expressed protein compliment of the genome [Wasinger, Cordwell et al. (1995) Electrophoresis 16: 1090-4]. Proteomics methods allow for the discovery of novel TAAs in an unbiased fashion. Common proteomics methods for discovery of novel TAAs and autoimmune autoantigens include SEREX (serological identification of antigens by recombinant expression cloning) [Krebs, Kurrer Sahin, Tureci and Ludewig (2003) Autoimmun Rev 2: 339-45; Tureci, Usener, Schneider and Sahin (2005) Methods Mol Med 109: 137-54; Tan, Low, Lim and Chung (2009) FEBS J 276: 6880-904; Heller, Zornig et al. (2010) Cancer Immunol Immunother 59: 1389-400; Stempfer, Syed et al. (2010) BMC Cancer 10: 627] and proteome microarrays (“chips”, commonly the dimensions of standard microscope slides, containing thousands of purified recombinant or tissue-derived proteins printed to their surface in an ordered array of microscopic spots, e.g. spots of 100 microns in diameter) [Robinson, DiGennaro et al. (2002) Nat Med 8: 295-301; Robinson, Steinman and Utz (2002) Arthritis Rheum 46: 885-93; Hudson, Pozdnyakova, Haines, Mor and Snyder (2007) Proc Natl Acad Sci USA 104: 17494-9; Babel, Barderas, Diaz-Uriarte, Martinez-Torrecuadrada, Sanchez-Carbayo and Casal (2009) Mol Cell Proteomics 8: 2382-95].

### SUMMARY OF THE INVENTION

In one embodiment, the present invention contemplates a method of diagnosing or determining prognosis of colorectal cancer (CRC) in an individual comprising: a) contacting a test sample from the individual with one or more target antigens, each comprising an antigen of Table I or fragments thereof comprising an epitope; and b) detecting binding of the one or more target antigens to one or more antibodies in the test sample, wherein the presence of the one or more antibodies bound against the one or more target antigens is indicative of colorectal cancer (CRC), or is indicative of CRC prognosis, aggressiveness, invasiveness or likelihood of recurrence. In one embodiment, the one or more target antigens are immobilized on a solid support. In one embodiment, the test sample is contacted with all of the target antigens of Table I or fragments thereof comprising an epitope. In one embodiment, the test sample is cells, tissues or body fluids. In one embodiment, the test sample is blood, plasma or serum.

In one embodiment, the present invention contemplates a method of detecting antibodies related to colorectal cancer (CRC) in an individual comprising: a) contacting a test sample from an individual with one or more target antigens of Table I; and b) detecting binding of the one or more target antigens to one or more antibodies in the test sample, wherein the presence of the one or more antibodies bound against the one or more target antigens is indicative of colorectal cancer (CRC). In one embodiment, the one or more target antigens are immobilized on a solid support. In one embodiment, the test sample is contacted with all of the target antigens of Table I. In one embodiment, the test sample is selected from the group consisting of cells, tissues or body fluids. In one embodiment, the test sample is selected from the group consisting of blood, plasma or serum.

In one embodiment, the present invention contemplates a method of diagnosing or determining prognosis of colorectal cancer (CRC) in an individual comprising: a) contacting a test sample from the individual with at least two or more target antigens, each comprising an antigen of Table II or fragments thereof comprising an epitope; and b) detecting binding of the at least two or more target antigens to one or more antibodies in the test sample, wherein the presence of the one or more antibodies bound against the at least two or more target antigens is indicative of colorectal cancer (CRC), or is indicative of CRC prognosis, aggressiveness, invasiveness or likelihood of recurrence. In one embodiment, the at least two or more target antigens comprise MAP4K4 of Table II. In one embodiment, the at least two or more target antigens comprise IGFBP3 of Table II. In one embodiment, the at least two or more target antigens are immobilized on a solid support. In one embodiment, the test sample is cells, tissues or body fluids. In one embodiment, the test sample is blood, plasma or serum.

In one embodiment, the present invention contemplates a method of detecting antibodies related to colorectal cancer (CRC) in an individual comprising: a) contacting a test sample from the individual with at least two or more target antigens, each comprising an antigen of Table II, wherein at least one of said target antigens is selected from the group consisting of MAP4K4 and IGFBP3; and b) detecting binding of the at least two or more target antigens to one or more antibodies in the test sample, wherein the presence of the one or more antibodies bound against the at least two or more target antigens is indicative of colorectal cancer (CRC). In one embodiment, the at least two or more target antigens are immobilized on a solid support. In one embodiment, the test sample is selected from the group consisting of cells, tissues or body fluids. In one embodiment, the test sample is selected from the group consisting of blood, plasma or serum.

In one embodiment, the present invention contemplates a method for identifying novel antigen/autoantigen biomarkers, said method comprising: a) determining the gene expression levels, expressed as mRNA or protein, of one, two or more genes in disease or disease-state individuals, tissues or cells and non-disease or non-disease-state individuals, tissues or cells; and b) comparing the level of expression of said one or more genes in said disease or disease-state individuals, tissues or cells to said non-disease or non-disease-state individuals, tissues or cells in order to identify candidate (potential) disease or disease-state associated antigens/autoantigens based on genes overexpressed or aberrantly expressed in said disease or disease-state individuals, tissues or cells versus said non-disease or non-disease-state individuals, tissues or cells; and c) assaying body fluid from individuals with said disease or disease-state, and from said non-disease or non-disease-state individuals, for antibodies/autoantibodies against said candidate antigens/autoantigens (gene products) to confirm or deny any valid disease or disease-state associated antigens/autoantigens from said candidates; and d) using said valid antigens/autoantigens in the diagnostic, prognostic, staging and/or therapeutic regimens for said disease or disease-state. In one embodiment, said gene expression levels are determined by measuring mRNA levels. In one embodiment, said mRNA levels are determined using DNA microarrays. In one embodiment, said gene expression levels are determined by measuring protein levels. In one embodiment, said gene expression levels are determined for 100 or more genes. In one embodiment, said gene expression levels are determined for 1,000 or more genes. In one embodiment, said gene expression levels are determined for 10,000 or more genes. In one embodiment, said disease is cancer. In one embodiment, said disease is colorectal cancer. In one embodiment, said disease-state is recurrent, aggressive or metastatic cancer and said non-disease-state is non-recurrent, non-aggressive or non-metastatic cancer. In one embodiment, said disease-state is recurrent, aggressive or metastatic colorectal cancer and said non-disease-state is non-recurrent, non-aggressive or non-metastatic colorectal cancer. In one embodiment, said antibodies or autoantibodies recognize tumor antigens/autoantigens. In one embodiment, said body fluid of step c) is blood, plasma or serum. In one embodiment, said antibody/autoantibody assay of step c) is performed using methods selected from the group consisting of immunohistochemistry, immunofluorescence, Western blot, dot blot, ELISA or bead based solid-phase immunoassay.

In one embodiment, the present invention contemplates a method for identifying antibodies related to cancer, said method comprising: a) comparing the gene expression level of one or more genes in cancer cells and normal cells; b) identifying one or more genes only activated in said cancer cells as compared to normal cells; c) assaying body fluid from at least one individual with said cancer type for antibodies to the gene product of said genes identified in step b); and d) identifying antibody reactive with at least one gene product assayed in step c). In one embodiment, gene expression levels are determined by measuring mRNA. In one embodiment, gene expression levels are determined by measuring protein. In one embodiment, said normal cells are from normal tissues. In one embodiment, said one or more genes identified in step b) are also not activated in non-recurrent cancer. In one embodiment, the method further comprises e) using the gene product reactive with said antibody of step c) to diagnose cancer in a person of unknown disease status.

In yet another embodiment, the present invention contemplates a method for identifying antibodies related to cancer, said method comprising: a) comparing the gene expression level of one or more genes in cancer cells and normal cells; b) identifying one or more genes activated more than 1.4 fold in said cancer cells as compared to normal cells; c) assaying body fluid from at least one individual with said cancer type for antibodies to the gene product of said genes identified in step b); and d) identifying antibody reactive with at least one gene product assayed in step c). In one embodiment, gene expression levels are determined by measuring mRNA. In one embodiment, gene expression levels are determined by measuring protein. In one embodiment, said normal cells are from normal tissues. In one embodiment, said body fluid is selected from the group consisting of serum and plasma. In one embodiment, the method further comprises e) using the gene product reactive with said antibody of step c) to diagnose cancer in a person of unknown disease status. In one embodiment, said one or more genes identified are activated more than 1.5 fold in said cancer cells as compared to normal cells. In one embodiment, said one or more genes identified are activated more than 1.8 fold in said cancer cells as compared to normal cells. In one embodiment, said one or more genes identified are activated more than 2.0 fold in said cancer cells as compared to normal cells. In one embodiment, said one or more genes identified in step b) are also activated more than 1.4 fold in said cancer cells as compared to non-recurrent cancer. In one embodiment, said cancer cells are from a solid tumor.

In still another embodiment, the present invention contemplates a method for identifying antibodies related to recurrent cancer, said method comprising: a) comparing the gene expression level of one or more genes in recurrent cancer cells and non-recurrent cancer cells; b) identifying one or more genes only activated in said recurrent cancer cells as compared to said non-recurrent cancer cells; c) assaying body fluid from at least one individual with said recurrent cancer for antibodies to the gene product of said genes identified in step b); and d) identifying antibody reactive with at least one gene product assayed in step c). In one embodiment, gene expression levels are determined by measuring mRNA. In one embodiment, gene expression levels are determined by measuring protein. In one embodiment, the method further comprises e) using the gene product reactive with said antibody of step c) to predict whether cancer in a person is recurrent.

In still another embodiment, the present invention contemplates a method for identifying antibodies related to recurrent cancer, said method comprising: a) comparing the gene expression level of one or more genes in recurrent cancer cells and non-recurrent cancer cells; b) identifying one or more genes activated more than 1.4 fold in said recurrent cancer cells as compared to said non-recurrent cancer cells; c) assaying body fluid from at least one individual with said recurrent cancer type for antibodies to the gene product of said genes identified in step b); and d) identifying antibody reactive with at least one gene product assayed in step c). In one embodiment, gene expression levels are determined by measuring mRNA. In one embodiment, gene expression levels are determined by measuring protein. In one embodiment, said body fluid is selected from the group consisting of serum and plasma. In one embodiment, the method further comprises e) using the gene product reactive with said antibody of step c) to predict whether cancer in a person is recurrent. In one embodiment, said one or more genes identified are activated more than 1.5 fold in said recurrent cancer cells as compared to non-recurrent cancer cells. In one embodiment, said one or more genes identified are activated more than 1.8 fold in said recurrent cancer cells as compared to non-recurrent cancer cells. In one embodiment, said one or more genes identified are activated more than 2.0 fold in said recurrent cancer cells as compared to non-recurrent cancer cells. In one embodiment, said recurrent cancer cells are from a solid tumor.

In one embodiment, the present invention relates to methods of using the novel tumor associated antigens/autoantigens (TAAs) mitogen-activated protein kinase kinase kinase kinase 4 (MAP4K4; Table I) and/or insulin-like growth factor-binding protein 3 (IGFBP3; Table I), or fragments thereof comprising an epitope, in the diagnostic, prognostic, staging and therapeutic regimens of colorectal cancer (CRC). The present invention also relates to methods of using a panel of TAAs (Table II), or fragments thereof comprising an epitope, in the diagnostic, prognostic, staging and therapeutic regimens of colorectal cancer (CRC).

The present invention further provides isolated antibodies/autoantibodies that bind specifically to the above-described polypeptide(s), or fragments thereof comprising an epitope. Antibodies/autoantibodies provided herein may be polyclonal or monoclonal, may be affinity purified, may be immobilized onto a solid support, and may be detectably labeled. The invention also provides methods for detecting the presence of CRC in an animal, preferably a human, comprising the steps of isolating a body fluid sample, preferably blood, serum or plasma, from the animal, incubating the sample with an isolated MAP4K4 and/or IGFBP3 polypeptide described above, and detecting the binding of antibodies/autoantibodies in the sample to the isolated polypeptide(s). The invention also provides alternative methods for detecting the presence of CRC in an animal comprising the steps of isolating a body fluid sample from the animal, preferably blood, serum or plasma, and immobilizing components of the sample on a solid support, contacting the immobilized sample components with an isolated polypeptide(s) described above under conditions favoring the formation of a complex between the sample components and isolated polypeptide(s), contacting the formed complex with an antibody that binds specifically to MAP4K4 and/or IGFBP3, and detecting the binding of the antibody to the complex. Cancers that may be diagnosed by the methods of the present invention include colorectal cancer (CRC). The present invention also provides methods of determining prognosis, disease stage and treatment regimens using the aforementioned methods of detecting autoantibodies against MAP4K4 and/or IGFBP3.

In a preferred embodiment, heterogeneous or homogenous immunoassays, single-plex or multiplex, are used to detect antibodies/autoantibodies present in body fluids directed against said TAAs. Other preferred embodiments of the present invention will be apparent to one of ordinary skill in light of the following drawings (Figures) and description of the invention, and of the claims.

An aspect of this invention, as illustrated in Experimental Examples 1-4, is the discovery of novel disease-associated antigens/autoantigens by: i) first performing gene expression analysis (measured as the level of mRNA or protein expressed), sometimes referred to as gene expression profiling (GEP), in disease or disease-state individuals, tissues or cells and non-disease or non-disease-state individuals, tissues or cells in order to identify candidate (potential) antigens/autoantigens associated with a given disease or disease-state, followed by ii) screening of blood/plasma/serum/bio-fluid from individuals with the targeted disease (or disease-state), and from control individuals without said disease or disease-state, for antibodies/autoantibodies against the candidate (potential) antigens/autoantigens, in order to discover any valid disease or disease-state associated antigens/autoantigens from said candidates. It is to be understood that such an approach is designed to significantly improve current methods of identifying antigens/autoantigens which can be used for the screening, diagnosing, monitoring or prognosing of a disease or disease-state associated with the formation of specific antigens/autoantigens, and can also potentially be used for treatment of such diseases/disease-states.

In one preferred embodiment, gene expression is assayed using genome-wide analysis of mRNA levels, for example with DNA microarray technology (commonly known to those skilled in the art), in the disease or disease-state tissue or cells versus non-disease or non-disease-state tissue or cells. Candidate disease or disease-state associated antigens/autoantigens are identified by their aberrant expression or overexpression in the disease or disease-state tissue or cells as compared to the non-disease or non-disease state tissue or cells. Candidate antigens/autoantigens are then validated by screening the blood/plasma/serum/body fluid of individuals with the disease or disease-state, and control individuals without the disease or disease state, against the candidate antigens/autoantigens in order to detect antibody/autoantibody reactivity with the candidate antigens/autoantigens (for example using immunoassays such as ELISA). In another preferred embodiment, gene expression analysis is performed by measuring protein levels, for example using proteomics technologies such as two-dimensional gel electrophoresis and/or liquid chromatography coupled mass spectrometry techniques (commonly known to those skilled in the art).

Several explanations have been proposed for the formation of a humoral immune response to tumor or autoimmune antigens/autoantigens, including aberrant expression, degradation, activation or cellular localization as well as mutations and protein misfolding [Casiano, Mediavilla-Varela et al. (2006) Mol Cell Proteomics 5: 1745-59; Rosen and Casciola-Rosen (2009) J Intern Med 265: 625-31; Tan, Low, Lim and Chung (2009) FEBS J 276: 6880-904; Casal and Barderas (2010) Mol Diagn Ther 14: 149-54]. It is not intended that the present invention be limited to any such mechanism.

Aberrant expression or overexpression of some TAAs in the diseased tissue has been established. For example, in one early study, candidate TAAs were identified for esophageal squamous cell carcinoma using SEREX (i.e. screening of patient serum for antibody/autoantibody reactivity to proteins/peptides), and subsequent gene expression analyses demonstrated that one of the TAAs, NY-ESO-1, is aberrantly expressed in a wide range of cancers [Chen, Scanlan et al. (1997) Proc Natl Acad Sci USA 94: 1914-8] Likewise, in another study, several TAAs for ovarian cancer were identified by screening patient serum for antibodies/autoantibodies using high density proteome microarrays, and again, subsequent gene expression analysis demonstrated that some TAAs were indeed overexpressed in the cancer tissue compared to that of healthy individuals [Hudson, Pozdnyakova, Haines, Mor and Snyder (2007) Proc Natl Acad Sci USA 104: 17494-9]. Similarly, another study, which reported the discovery novel TAAs for colorectal cancer [Babel, Barderas, Diaz-Uriarte, Martinez-Torrecuadrada, Sanchez-Carbayo and Casal (2009) Mol Cell Proteomics 8: 2382-95], also identified the TAAs by serum screening against high density protein microarrays, followed by gene expression analysis of the discovered TAAs.

Another report notes that it is “ . . . the first to combine genome-wide expression signatures and comprehensive seroreactivity patterns toward a more complete view on tumor immunology . . . ” [Keller, Ludwig, Comtesse, Henn, Steudel, Lenhof and Meese (2009) Gene Ther 16: 184-9]. However, analogous to the aforementioned studies, this work began with a known set of TAAs and next performed genome-wide gene expression analysis to confirm aberrant or overexpression of the genes corresponding to the known TAAs. In contrast, in this invention, genome-wide gene expression analysis was used for the first time to guide the subsequent discovery (and validation) of TAAs using blood-based antibody/autoantibody assays.

In one embodiment, the present invention contemplates immunizing humans or animals with MAP4K4 of Table II and/or IGFBP3 of Table II. Such immunizing can comprise an initial immunization together with later booster immunizations, until circulating antibody is detectable.

### DESCRIPTION OF THE FIGURES

FIG. 1: mRNA Expression Analysis of MAP4K4 in Recurrent (R) and Non-Recurrent (NR) CRC Patient (Tumor) Samples Using DNA Microarrays. Data are shown as a Box-and-Whisker plot. R=Recurrent CRC patient samples and NR=Non-Recurrent CRC patient samples. Note that the y-axis (log₂ fluorescence intensity) is in arbitrary units.

FIG. 2: Proteome Microarray (ProtoArray®) Analysis of the Novel Tumor Autoantigen Human MAP4K4 on 95 Distinct Serum Samples. Autoantibody fluorescence signal intensity (“Normalized Array Signal”) for each of the patient serum samples is shown for the novel tumor autoantigen MAP4K4 (data are quantile normalized across the entire microarray set on a per lot basis). Serum samples are denoted by their “Serum ID” whereby the prefix CRC=Colorectal Cancer; N=Normal (Healthy Individuals); PBC=Primary Biliary Cirrhosis; SjS=Sjögren's Syndrome; SLE=Systemic Lupus Erythematosus. Two microarray lots were run and are shown in separate graphs. The red box denotes the overall CRC patient cohort, green the normal patients and blue the autoimmune patients.

FIG. 3: ELISA Validation of the Novel Tumor Autoantigen Human MAP4K4. Purified human recombinant MAP4K4 protein was bound directly to the polystyrene microtiter ELISA plate surface and used to assay patient serum for the presence of autoantibodies. RLU=Relative Luminescence Units of the ELISA assay readout. Serum samples are denoted by their “Serum ID” whereby the prefix CRC=Colorectal Cancer and N=Normal (Healthy Individuals). The red horizontal line indicates the diagnostic scoring cutoff. The red box denotes the overall CRC patient cohort and green box the normal patients.

FIG. 4: Gene Expression-Guided Discovery of Novel Colorectal Cancer (CRC) Autoantigens MAP4K4 and IGFBP3 Using Multiplexed Bead-Based System. Protein autoantigens were bound directly to the VeraCode™ carboxyl beads and used to assay patient serum or plasma for the presence of autoantibodies. (A-D) TAA candidates selected for serum/plasma screening based on prior gene expression analysis (images not shown). Known/Published TAAs selected for serum/plasma screening based on the scientific literature. Individual proteins were as follows: (A.) IGFBP3; (B.) MAP4K4; (C.) IGFBP5; (D.) SULF1. MFI=Mean Fluorescence Intensity of the BeadXpress™ instrument readout. Individual patient samples are denoted on the x-axis whereby the prefix CRC=Colorectal Cancer and N=Normal (Healthy Individuals). The red horizontal line indicates the diagnostic scoring cutoff (whereas the dark red vertical bars are positive samples). The overall CRC and normal patient cohorts are also labeled below the x-axis.

### EXPERIMENTAL

### Example 1

### Gene Expression Analysis of MAP4K4 in Colorectal Cancer

Gene Expression Analysis of Recurrent vs. Non-Recurrent CRC

The CRC gene expression dataset was exclusively licensed from Ananomouse Corporation (Cambridge, Mass.) and was produced by whole-genome DNA microarray analysis as follows: The tumor tissue was assayed on the industry standard for oligonucleotide microarrays, the Affymetrix (Santa Clara, Calif.) GeneChip® Human Genome U133 Plus 2.0 array. Analytics were performed utilizing Praxis™ (Ananomouse Corporation, Cambridge, Mass.), a bioinformatics analysis software tool. A component of Praxis™ implements stringent quality assurance metrics to ensure only the highest-quality arrays continue on to the final analysis, which reduces intra-class variability and maintains high signal-to-noise ratios. A power analysis based on preliminary data determined 25 samples were required per class (recurrent and non-recurrent CRC) to achieve greater than 90% power to detect a true difference in expression between classes of at least 0.5-fold, when group standard deviations are less than 0.80 (97.5% of the probes on the microarray) with a false discovery rate of 0.05.

Once the Praxis™ software tool completed normalization and differential expression measures on the microarrays, it conducted gene set enrichment analyses of the data and incorporated comprehensive clinical history reports for each patient sample in order to create a gene expression list that cross-correlated both genotypic and phenotypic features. This list of genes differentially expressed between the two classes (recurrent and non-recurrent CRC) was further refined by applying a meta-analysis of publicly accessible microarray data to add statistical weight and significance to genes that were similarly differentiated. Additionally, for detecting tumor associated antigen/autoantigen (TAA) candidates, public gene expression data for healthy patient tissue and tissues of other cancers was also used in the meta analysis. Importantly, the meta-analysis was not used to remove or add genes to the list, but simply to rank and prioritize them.

### Results:

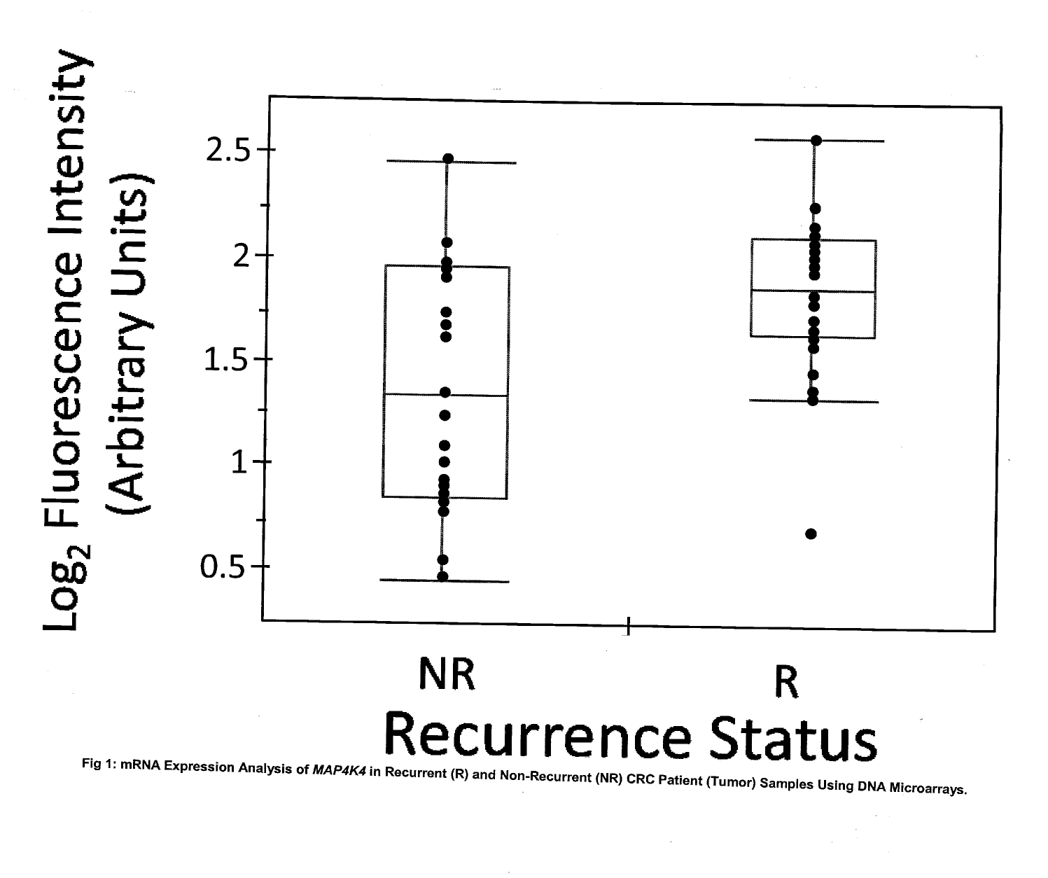

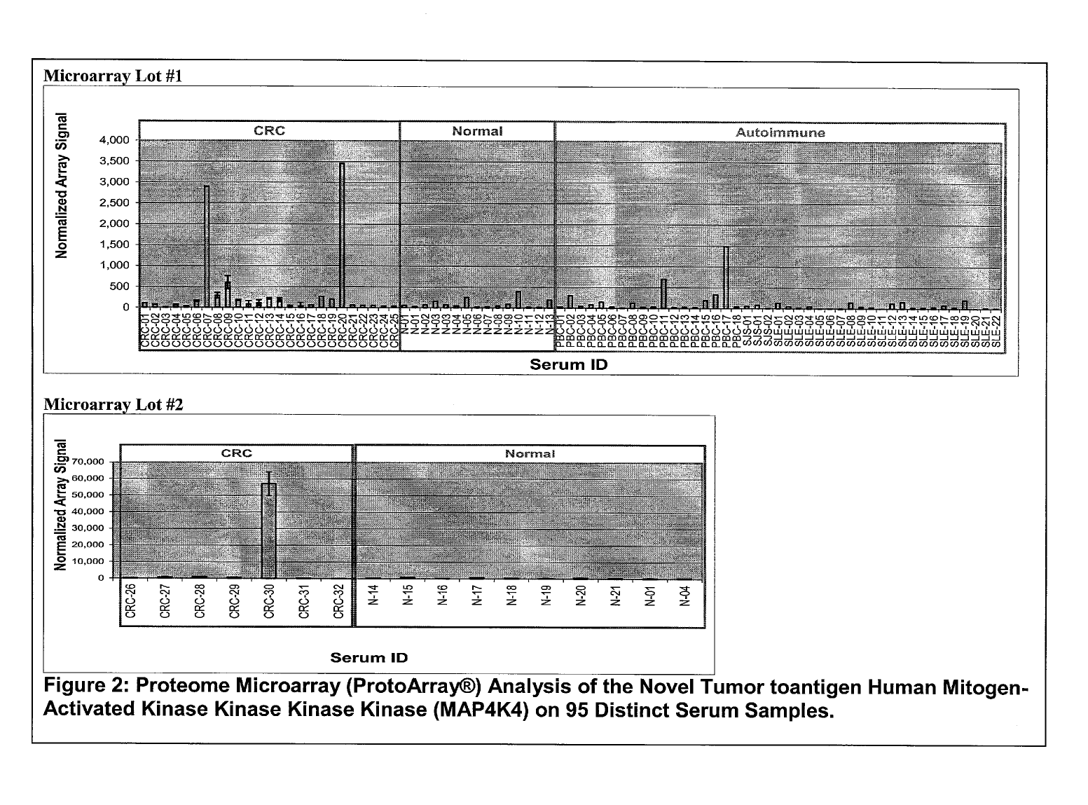

Genes that present the most promising targets as possible TAA biomarkers are those that are only activated in tumor tissue (as compared to normal tissue), and are also up-regulated in the recurrent class of patients. A preliminary statistical ranking of the data according to these parameters placed MAP4K4 at the top of the list. FIG. 1 shows the gene expression pattern of MAP4K4 in the recurrent and non-recurrent CRC cohorts based on the aforementioned DNA microarray analysis, indicating it is more highly expressed in the recurrent cohort (note that the y-axis, log₂ fluorescence intensity, is in arbitrary units). With respect to gene expression and prediction of CRC recurrence, MAP4K4, in conjunction with three other top-ranking differentially expressed genes (both up- and down-regulated), correctly identified the prognostic class of patients in an independent cohort with a 97% statistical accuracy.

Based on these results, further investigation of MAP4K4 as a TAA for CRC were also performed (see subsequent Experimental Examples).

### Example 2

### Gene Expression-Guided Proteome Microarray Based Analysis and Discovery of the Novel Colorectal Cancer (CRC) Autoantigen MAP4K4

The human MAP4K4 novel TAA was analyzed using a high density protein microarrays, to detect autoantibodies in the sera of CRC patients as well as healthy (normal) and autoimmune disease patient controls. As discussed further in the Results section of this Example, this discovery of MAP4K4 as a TAA was guided and facilitated by prior gene expression analysis (see Example 1). It should also be noted that while MAP4K4 was not previously known as a TAA, the mitogen activated protein kinase (MAPK) cell-signaling pathway, and more specifically, MAP4K4 gene expression, have also been associated with CRC in the scientific literature [Hao, Chen, Sui, Si-Ma, Li, Liu, Li, Ding and Li (2010) J Pathol 220: 475-89; Lascorz, Forsti et al. (2010) Carcinogenesis 31: 1612-9].

### Serum Screening on Microarrays

Patient sera were screened against commercial human proteome microarrays comprised of ˜8,000 unique human recombinant (eukaryotically expressed) proteins printed in duplicate at high density to a “chip” the size of a standard microscope slide (Human ProtoArray® v4.0, Invitrogen, Carlsbad, Calif.) [Sheridan (2005) Nat Biotechnol 23: 3-4]. Microarrays were performed according to the manufacturer's instructions. Microarrays were imaged on an ArrayWoRx^{e} BioChip fluorescence reader (Applied Precision, LLC, Issaquah, Wash.) using the appropriate standard built-in filter sets. Image analysis and data acquisition was performed using the GenePix Pro v6.1 software package (Molecular Devices, Sunnyvale, Calif.) according to the instructions of the microarray manufacturer (Human ProtoArray® v4.0, Invitrogen, Carlsbad, Calif.).

95 different serum samples from normal individuals and patients with various diseases were individually screened against the proteome microarrays in order to detect the presence of autoantibodies against the arrayed proteins (potential autoantigens). For this, 2 different lots of microarrays were used in 2 sequential studies. The composition of the entire patient population was as follows: Microarray Lot #1 (80 unique samples)-25 colorectal cancer (CRC) patients versus 55 non-CRC control samples [13 normal, 18 Primary Biliary Cirrhosis (PBC), 22 systemic lupus erythematosus (SLE), 2 Sjögrens syndrome (SjS)]. Microarray Lot #2 (15 unique samples)-7 more CRC and 8 more normal patients. Due to some serum samples being run multiple microarrays, the total number of microarrays run was 100. The normal sera were approximately age and gender matched to the CRC cohort. Archived sera were obtained from the repositories of the following sources: 27 CRC sera were from Asterand Inc. (Detroit, Mich.); all normal sera and 5 CRC sera were from ProMedDx, LLC (Norton, Mass.); Dr. Donald Bloch, M.D., Center for Immunology and Inflammatory Diseases, Massachusetts General Hospital, Assistant Professor of Medicine, Harvard Medical School provided 12 of the SLE sera as well as the SjS and PBC sera; remaining SLE sera were from Bioreclamation Inc. (Hicksville, N.Y.).

All but 5 of the CRC samples were stage T2 or T3 (AJCC staging) all non-metastatic. Of the remaining 5 CRC samples, 1 was T1 non-metastatic, 1 was of unknown staging, 1 was T2 metastatic, 1 was T3 metastatic and 1 was T4 metastatic.

### Biostatistical Analysis of Microarray Data

The biostatistical methods used were the standard approaches provided by the microarray manufacturer in the form of the ProtoArray® Prospector v4.0 software package (Invitrogen, Carlsbad, Calif.) using the Immune Response Profiling (IRP) add-on [Hudson, Pozdnyakova, Haines, Mor and Snyder (2007) Proc Natl Acad Sci USA 104: 17494-9]. The software uses the M-Statistics algorithm: This approach uses quantile normalized microarray data and performs a pairwise t-test for each protein between the two patient cohorts (i.e. CRC group and the control group, corresponding to all non-CRC patients in this case). This algorithm also estimates the autoantigen prevalence in the various patient cohorts (sensitivity and specificity) based on cutoffs set by the quantile normalized data.

### Results:

The novel TAA biomarker for CRC, mitogen-activated protein kinase kinase kinase kinase 4 (MAP4K4/HGK), is listed in Table I (see Table III for protein used in this Experimental Example) (SEQ ID NO:1). Quantile normalized microarray data (normalized autoantibody signal intensity) for all 95 samples are shown in FIG. 2 for MAP4K4. In summary, the presence of serum autoantibodies against the MAP4K4 autoantigen is correlated with the CRC cohort, showing a modest M-Statistics p-value of 0.09 as well as a sensitivity of 11.1% and a specificity of 98.3% (determined from Microarray Lot #1 using ProtoArray® Prospector v4.0 software). These performance traits are typical for a TAA, as it is well established in the literature that a single TAA biomarker (i.e. autoantibody responses to the TAA) will rarely yield a diagnostic sensitivity exceeding 10-15%, although they are of generally very high specificity [Zhang, Casiano, Peng, Koziol, Chan and Tan (2003) Cancer Epidemiol Biomarkers Prev 12: 136-43; Casiano, Mediavilla-Varela et al. (2006) Mol Cell Proteomics 5: 1745-59; Belousov, Kuprash et al. (2008) Biochemistry (Mosc) 73: 562-72].

Of the 3 MAP4K4-positive CRC patients, 2 were stage T2N0M0 (CRC-07 and CRC-20) and 1 was T3N0M0 (CRC-30).

Importantly, MAP4K4 was not a top ranking candidate TAA for CRC based on the proteome microarray M-Statistics. In fact, when the microarray data are ranked by statistical significance (M-Statistics p-value; CRC vs. all non-CRC from Microarray Lot #1), MAP4K4 was tied at the 388^{th} ranking TAA for CRC. Focus was only directed to MAP4K4 within this full protein microarray dataset based on prior gene expression analysis (Example 1), and MAP4K4 was pursued for further validation based on this (see subsequent Examples).

Finally, in addition to diagnostics, it is anticipated that the MAP4K4 TAA will be useful in determining CRC prognosis, outcome, recurrence and/or aggressiveness since separate gene expression analysis indicates MAP4K4 overexpression is associated with recurrent/aggressive CRC (see Example 1).

### Example 3

### Validation of Novel Colorectal Cancer (CRC) Autoantigen MAP4K4 Using an ELISA

The human MAP4K4 TAA was validated using an Enzyme-Linked Immunosorbent Assay (ELISA) to detect autoantibodies in the sera of CRC and healthy (normal) patients.

### Enzyme-Linked Immunosorbent Assay (ELISA) of Autoantigen

Note that some of the CRC and normal patient sera used in the ELISA were the same as used on the ProtoArray® microarrays, while others were not. CRC and normal sera were from Asterand Inc. (Detroit, Mich.), ProMedDx, LLC (Norton, Mass.) and the Ontario Institute of Cancer Research (OICR). A total of 47 normal and 47 CRC sera were used.

CRC sera were an approximate 50:50 distribution of a) stage T2 or T3 (AJCC staging) non-metastatic and b) stage T3 or T4 metastatic.

Human MAP4K4 recombinant protein expressed in insect cells and purified by its N-terminal GST fusion tag was purchased from Invitrogen (Carlsbad, Calif.; catalog number PV3687). 384-well white opaque, flat bottom, untreated polystyrene microtiter plates (Microlite 1+; Thermo Fisher Scientific Inc., Waltham, Mass.) were coated overnight with 30 μL per well of 0.5 μg/mL recombinant MAP4K4 protein diluted in PBS (48 mM sodium phosphate, pH 7.5, 100 mM NaCl). Plates were then washed 6× in TBS-T (wells filled to maximum) on an ELx405 Select Robotic Plate Washer (BioTek, Winooski, Vt.). All plate washes were performed in this manner unless noted otherwise. All other liquid handling steps for the ELISA were performed using a Matrix PlateMate 2×3 liquid handling robot (Thermo-Fisher).

Plates were next blocked for 30 min at 90 μL/well in 1% BSA (w/v) in TBS-T. The block solution was removed from the plates and serum samples (diluted at 1/1,000 in 1% BSA (w/v) in TBS-T) were added at 30 μL/well and shaken for 30 min at room temperature. To avoid contamination of the robotic plate washer with human serum, plates were subsequently washed 3× by using the aforementioned Matrix PlateMate 2×3 liquid handler to add and remove the TBS-T washes (wells filled to maximum). Plates were then additionally washed 6× in the robotic plate washer as described earlier in this Example. Bound autoantibody was detected using 30 μL/well of a mouse anti-[human IgG]-HRP labeled monoclonal secondary antibody (Jackson ImmunoResearch Laboratories, Inc, West Grove, Pa.) diluted 1/20,000 in 1% BSA/TBS-T. Plates were shaken for 30 min. The solutions were then manually dumped from the plates by inversion followed by vigorous patting of the plates inverted on a dry paper towel to remove residual fluid. Plates were then washed in the robotic plate washer as described earlier in this Example. Chemiluminescence signal was generated by the addition of 30 μL/well of SuperSignal ELISA Pico Chemiluminesence Substrate (Pierce Biotechnology brand from Thermo Fisher Scientific Inc., Rockford, Ill.). Plates were developed by shaking for 15 min and then read on a VictorLight luminescence plate reader (Wallac/PerkinElmer Life and Analytical Sciences, Inc., Boston, Mass.).

### Results:

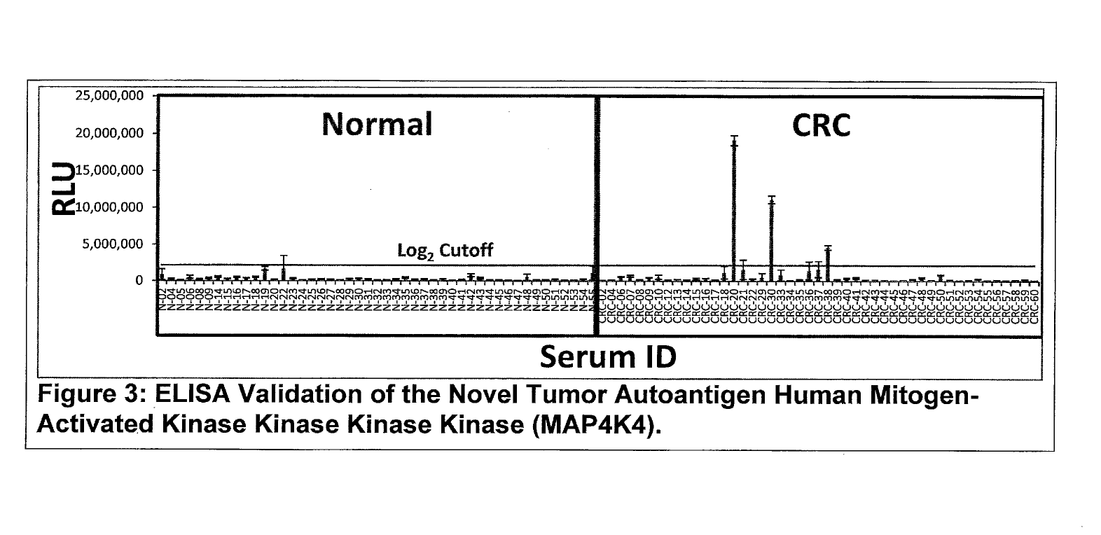

To calculate cutoffs, the ELISA values were log₂-transformed (to achieve Gaussian distribution of the data) and the standard deviation across the normal patient cohort was calculated. Results are shown in FIG. 3. A diagnostic scoring cutoff set at 3 standard deviations above the mean for the normal patient cohort (log₂ data) yields 6% sensitivity for CRC detection and 100% specificity with these samples. This method of setting cutoffs is commonly used for autoantibody immunoassays (e.g. [Liu, Wang, Li, Xu, Dai, Wang and Zhang (2009) Scand J Immunol 69: 57-63]). Serum samples CRC-20 (Stage T2N0M0) and CRC-30 (Stage T3N0M0) which were positive on the ProtoArray® microarrays were confirmed as positive in the ELISA, an additional serum, CRC-38 (not screened on ProtoArray® microarrays), was also detected as MAP4K4 positive in the ELISA (Stage T4N2M1). Note that this is an expected result because the sensitivity of any single TAA (autoantibody) biomarker rarely exceeds 10-15% [Zhang, Casiano, Peng, Koziol, Chan and Tan (2003) Cancer Epidemiol Biomarkers Prev 12: 136-43; Casiano, Mediavilla-Varela et al. (2006) Mol Cell Proteomics 5: 1745-59; Belousov, Kuprash et al. (2008) Biochemistry (Mosc) 73: 562-72].

### Example 4

### Gene Expression-Guided Discovery of Novel Colorectal Cancer (CRC) Autoantigens MAP4K4 and IGFBP3 Using Multiplexed Bead-Based Immunoassay

A candidate list of potential TAAs was first generated based on the genome-wide gene expression analysis described in Example 1. The corresponding recombinant proteins for 4 candidate TAAs from this list were subsequently selected for the screening of patient serum/plasma samples for autoantibody reactivity. As a comparison, 6 TAAs, known/reported in the scientific literature were also chosen for analysis. To perform these experiments, multiplexed immunoassays were done using the VeraCode™ micro-bead platform technology using specific modifications developed by AmberGen to bind the antigen to the bead surface.

Gene expression derived candidate TAAs were: MAP4K4, IGFBP3, IGFBP5 and SULF1 (note that SULF1 was also very recently reported in the scientific literature as a possible TAA for CRC based on phage microarrays [Babel, Barderas, Diaz-Uriarte, Moreno, Suarez, Fernandez-Acenero, Salazar, Capella and Casal (2011) Mol Cell Proteomics 10: M110 001784]).

Known/reported TAAs were p53, IGF2BP2, Cyclin B1, C-Myc, STK4 and NUCB1 [Koziol, Zhang, Casiano, Peng, Shi, Feng, Chan and Tan (2003) Clin Cancer Res 9: 5120-6; Zhang, Casiano, Peng, Koziol, Chan and Tan (2003) Cancer Epidemiol Biomarkers Prev 12: 136-43; Chen, Lin, Qiu, Peng, Looi, Farquhar and Zhang (2007) Int J Oncol 30: 1137-44; Babel, Barderas, Diaz-Uriarte, Martinez-Torrecuadrada, Sanchez-Carbayo and Casal (2009) Mol Cell Proteomics 8: 2382-95; Babel, Barderas, Diaz-Uriarte, Moreno, Suarez, Fernandez-Acenero, Salazar, Capella and Casal (2011) Mol Cell Proteomics 10: M110 001784].

### Attachment of Recombinant Proteins to VeraCode™ Beads

Human recombinant proteins IGF2BP2, IGFBP3 and STK4 were purchased from Sino Biological Inc (Beijing, China); Human recombinant protein MAP4K4 was purchased from Invitrogen (Carlsbad, Calif.); Human recombinant protein TP53 (p53) was purchased from Santa Cruz (Santa Cruz, Calif.); Human recombinant proteins CCNB1, IGFBP5 and NUCB1 were purchased from Abcam (Cambridge, Mass.); Human recombinant protein SULF1 was from Novus Biologicals (Littleton, Colo.); Human recombinant C-Myc was purchased from StemRD (Burlingame, Calif.).

Proteins were passed over a PD SpinTrap G-25 Column (GE Healthcare Life Sciences) to remove incompatible buffer components. First, the PD SpinTrap G-25 columns were equilibrated by adding 300 μL 1×PBS buffer and spinning for 1 minute at 800×g. Then 70-130 μL of the manufacturer supplied protein was applied and eluted by centrifuging for 2 minutes at 800×g. Following the desalting (buffer exchange), 5×PBS was added to the beads to bring up the total buffer to 1×PBS to ensure an adequate buffering capacity of the protein for the subsequent bead attachment steps. Note that for some proteins, the column buffer exchange step was omitted and the manufacturer supplied proteins were simply supplemented to 1×PBS from a 5× stock or supplemented to 1× or 2×MES Buffer (1×=0.1 M MES, pH 4.7, 0.9% NaCl) from a 10× stock. Protein concentration used for subsequent bead attachment was approximately 0.1 μg/μL.

Recombinant proteins were attached to carboxyl-modified VeraCode™ beads (Illumina, San Diego, Calif.) by a two-step method. VeraCode™ beads are 240×28 micron, holographically encoded, glass micro-cylinders with a carboxylated surface chemistry. First, 10,000 to 40,000 VeraCode™ beads were washed 3×800 μL with MES Buffer (0.1 M MES, pH 4.7, 0.9% NaCl) by sequential mixing, pelleting the beads by brief and gentle spinning (or allowing beads to settle by gravity) and removing the supernatant (wash buffer) by manual pipetting, being careful not to lose the bead pellet. All washes were performed in this manner unless otherwise indicated. After discarding the final wash, 200 μL of Sulfo-NHS Buffer (1 mg/mL in MES Buffer; prepared immediately prior to use) was added to each washed bead pellet. Beads were mixed immediately and briefly. 200 μL of EDC Buffer (1 mg/mL in MES Buffer; prepared immediately prior to use) was immediately added to each sample (containing both beads and Sulfo-NHS Buffer) and immediately mixed to combine. Following incubation for 1 hour with gentle mixing, the beads were washed 3×800 μL briefly with MES Buffer and then 1×800 μL quickly with 1×PBS (for proteins in MES Buffer, this PBS wash was omitted). The protein coupling reaction immediately followed, in which 10-40 μg of the previously prepared protein was added to the beads, mixed, and incubated for 1 hour at room temperature with mixing. Beads were then spun down, and the protein solution was removed. The beads were washed 2×800 μL briefly with 1% BSA (w/v) in TBS-T before discarding the wash and incubation with an additional 400 μL of 1% BSA (w/v) in TBS-T for 30 minutes. Beads were then washed briefly 1× with 800 μL of PBS-1M NaCl, 1×30 min with 400 μL of PBS-1M NaCl (with shaking) and then 2× briefly with 800 μL TBS-T. Beads were stored in TBS-T at 4° C.

### Serum Probing on VeraCode™ Beads

CRC and normal, sera and plasma, were from Asterand Inc. (Detroit, Mich.), ProMedDx, LLC (Norton, Mass.), the Ontario Institute of Cancer Research (OICR) and Analytical Biological Services Inc. (Wilmington, Del.). A total of 77 normal and 92 CRC sera and plasma were used.

CRC patient samples were an approximate 50:50 distribution of a) stage T2 or T3 (AJCC staging) non-metastatic and b) stage T3 or T4 metastatic.

To perform a multiplexed bead experiment, beads with the different proteins, each identifiable by a unique holographic barcode, were pooled into a round bottom 96-well polypropylene microtiter plate. Human plasma samples (diluted at 1/50 in 1% BSA [w/v] in TBS-T) were added at 100 μL/well and shaken for 30 minutes at room temperature. Samples were removed and beads were washed 6×250 μL briefly with 1% BSA (w/v) in TBS-T. Beads were then probed with 100 μL of an Anti-Human IgG Fluorescent (Dylight 649) Secondary Antibody diluted to 10 μg/mL (−65 nM) in 1% BSA (w/v) in TBS-T. Probing was for 30 minutes with mixing (1,200 rpm). The probe solution was removed and discarded, and the beads washed 6×250 μL briefly with TBS-T. The final wash solution was discarded, leaving the bead pellets and a small residual liquid volume in the wells of the readout plate (˜70 μL). Beads were scanned using the BeadXpress™ reader (Illumina, San Diego, Calif.).

### Results:

To process the data resulting from these TAA screening experiments, the mean fluorescence intensity (MFI) for each protein in each patient sample was used (an average of 30 replicate beads was used for each bead species in each patient sample). Known-positive sample-protein pairs were included in each assay as controls. Inter-assay normalization was performed based on data from 3 known-positive sample-protein pairs. To calculate cutoffs, in order to score samples as autoantibody positive or negative, the normalized MFI values were log₂-transformed (to achieve proper Gaussian distribution of the data) and the standard deviation across the normal patient cohort was calculated. The scoring cutoff was set at 3 standard deviations above mean of the normal patient cohort (4 standard deviations for IGFBP3).

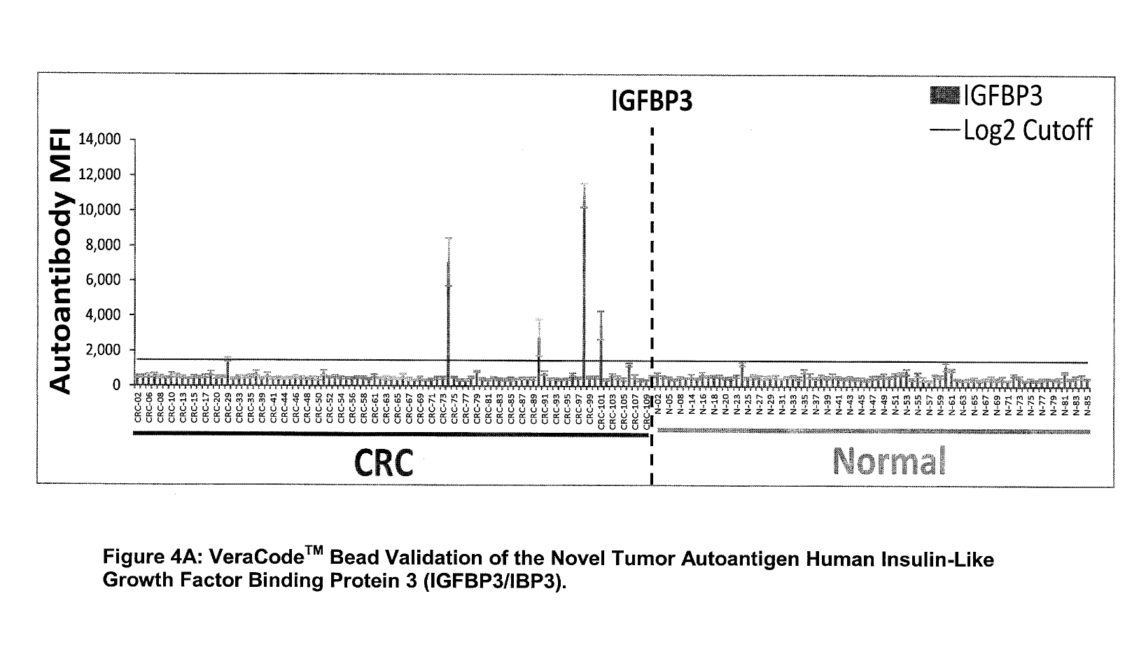

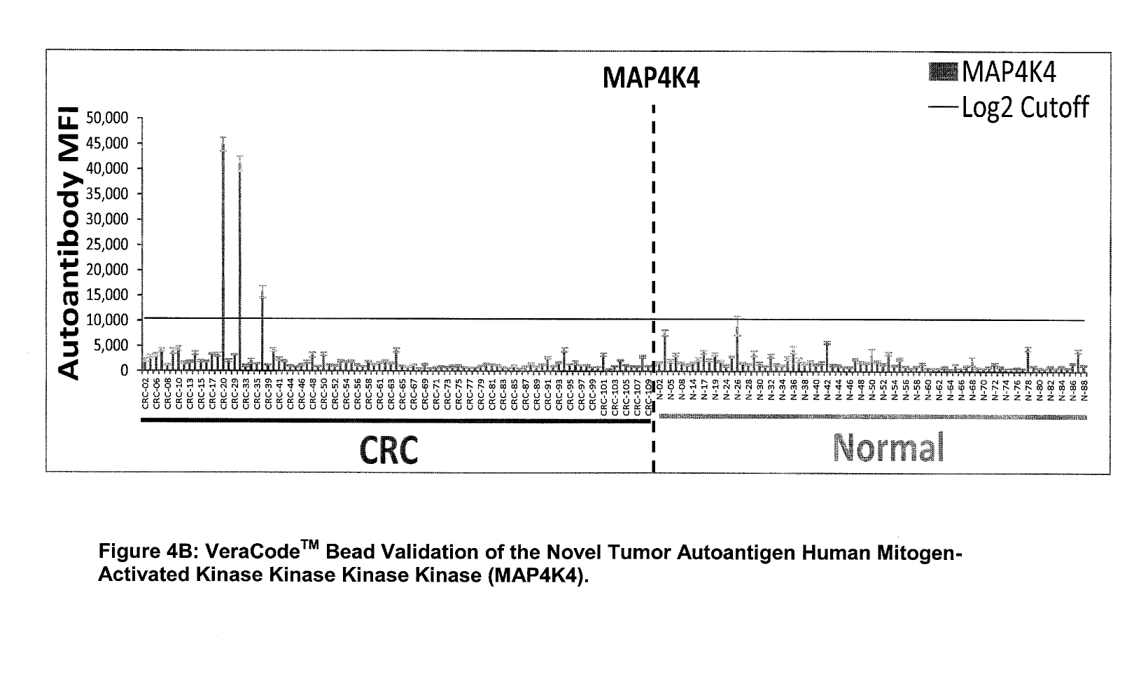

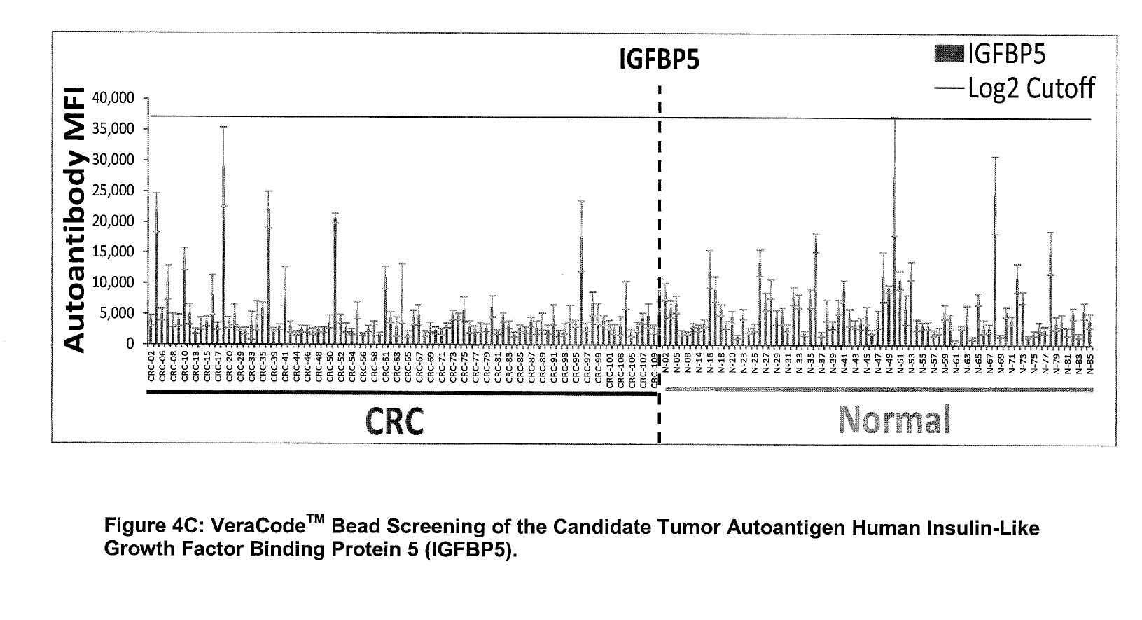

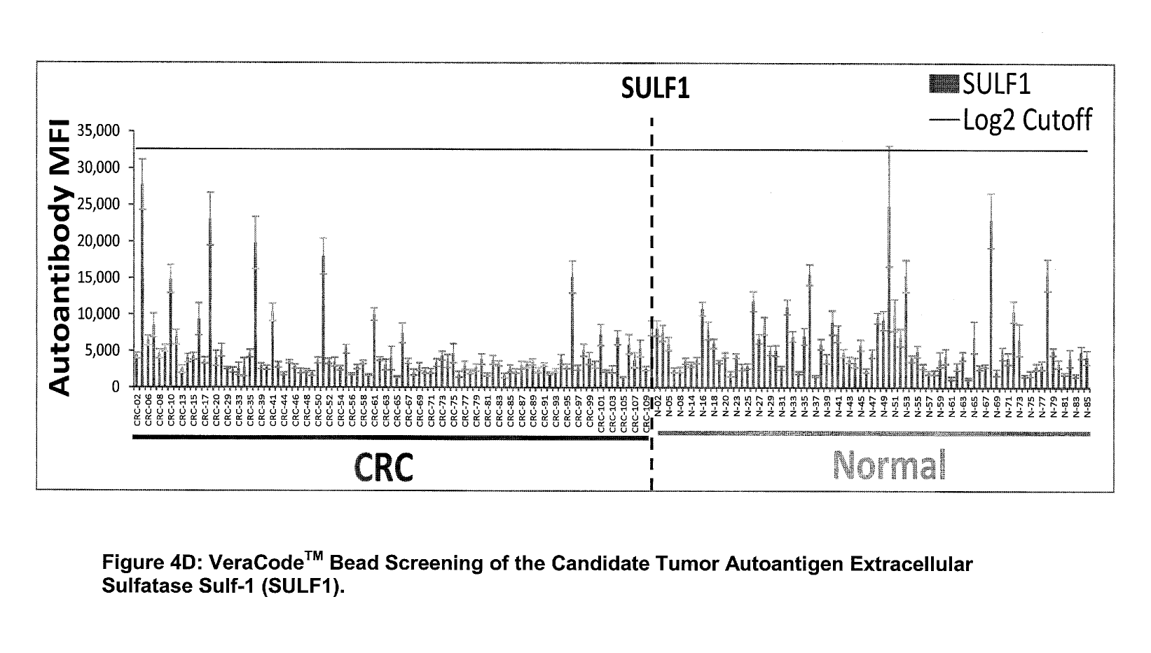

Results are shown in FIGS. 4A-J for all 10 TAAs screened in the present Example. The graphs in FIGS. 4A-J are not log₂ transformed data, but the cutoff and scoring was based on log₂ data. The error bars represent the intra-assay bead-to-bead variance in fluorescence intensity within each sample-protein pair (i.e. variance of replicate beads).

Of the 4 candidate TAAs which were identified from prior gene expression analysis (see Example 1 for example gene expression analysis), mitogen-activated protein kinase kinase kinase kinase 4 (MAP4K4) and insulin-like growth factor-binding protein 3 (IGFBP3) both showed significant association with CRC (FIGS. 4A and 4B). These novel TAAs are listed in Table I. IGFBP3 was 5% sensitive and 100% specific for CRC (positive predictive value of 100%) and MAP4K4 was 3% sensitive and 100% specific (positive predictive value of 100%). Although of relatively low sensitivity, these performance traits are typical for TAAs, as it is well established in the literature that a single TAA biomarker (i.e. autoantibody responses to the TAA) will rarely yield a diagnostic sensitivity exceeding 10-15%, although they are of generally very high specificity [Zhang, Casiano, Peng, Koziol, Chan and Tan (2003) Cancer Epidemiol Biomarkers Prev 12: 136-43; Casiano, Mediavilla-Varela et al. (2006) Mol Cell Proteomics 5: 1745-59; Belousov, Kuprash et al. (2008) Biochemistry (Mosc) 73: 562-72; Reuschenbach, von Knebel Doeberitz and Wentzensen (2009) Cancer Immunol Immunother 58: 1535-44]. Conversely, IGFBP5 and SULF1 showed no significant association with CRC in this analysis (0% sensitivity for CRC; FIGS. 4C and 4D). Therefore, the overall TAA validation success rate for this study was 50% using the method of a) candidate TAA selection by gene expression analysis followed by b) validation using blood-based immunoassays of the candidate recombinant TAA proteins.

As a basis for comparison to the aforementioned gene expression guided approach, several TAAs were tested which were previously known/reported in the scientific literature. Of these, IGF2BP2 and p53 showed significant association with CRC (FIGS. 4E and 4F). IGF2BP2 was 3% sensitive and 99% specific for CRC (positive predictive value of 75%), while p53, the most robust TAA of all those tested, was 16% sensitive and 100% specific (positive predictive value of 100%). Conversely, CCNB1, C-Myc, NUCB1 and STK4 showed no significant association with CRC in this analysis (all 0% sensitivity for CRC except STK4, which showed equal numbers of positives in the CRC and normal patient cohorts for a positive predictive value of 50%; see FIGS. 4G-J). Therefore, the overall TAA validation success rate for this study was 33% using the method of a) TAA selection from literature reports followed by b) validation using blood-based immunoassays of the recombinant TAA proteins.

Critically, using a panel of 4 TAAs comprising MAP4K4, IGFBP3, IGF2BP2 and p53, a composite sensitivity of 27% for CRC was achieved with 99% specificity (positive predictive value of 96%). This additive benefit of using multiple TAA biomarkers stems from their low redundancy, whereby, of the 25 CRC patients positive for at least 1 of these 4 TAAs, only 1 CRC patient was positive for multiple TAAs (that is, overlap of p53 and IGF2BP2 on 1 CRC patient).

AJCC tumor staging of the CRC patients which were positive for IGFBP3 was as follows (note that staging information was available for 3 of the 5 positive patients): 1 each at T2NXM0, T3N0MX and T4N0M0. Staging of all CRC patients which were positive for MAP4K4 was as follows: 1 each at T2N0M0, T3N0M0 and T4N2M1. Staging of all CRC patients which were positive for IGF2BP2 was as follows: 1 at T3N1M1 and 2 at T4N2M1. Staging of all CRC patients which were positive for p53 was as follows: 4 at T2N0M0, 1 at T2N0MX, 2 at T2NXM0, 4 at T3N0M0, 1 at T3N1M0, 1 at T4N2M0 and 2 at T4N2M1.

Importantly, separate gene expression analysis of MAP4K4 and IGFBP3 indicates they are more highly expressed in aggressive/recurrent CRC versus non-recurrent CRC (e.g. see Example 1 for MAP4K4 example). Thus, in addition to diagnostics, it is anticipated that the novel MAP4K4 and IGFBP3 autoantigens will be useful in determining CRC prognosis, outcome, recurrence and/or aggressiveness. The possibility also exists that the tested known/reported TAAs p53 and IGF2BP2 may also be associated with CRC recurrence. To this point, for all CRC patient samples tested for which recurrence status was known via 5 year follow-up (14 recurrent and 10 non-recurrent in this sample set), IGF2BP2 was positive in 14% of the recurrent patients and 0% of non-recurrent, MAP4K4 in 7% and 0% respectively, and IGFBP3 in 14% and 10% respectively, suggesting a possible association of these markers with CRC recurrence. Conversely, p53 was positive in 7% of the recurrent patients and 10% of non-recurrent.

TABLE I
Novel Tumor Autoantigens for CRC.

| Gene | | |
|---|---|---|
| Symbol | Description | Alternative Names or Synonyms |
| MAP4K4 | Mitogen-activated | HPK/GCK-like kinase HGK |
| | protein kinase kinase | MAPK/ERK kinase kinase kinase 4 |
| | kinase kinase 4 | MEK kinase kinase 4 |
| | | MEKKK 4 |
| | | Nck-interacting kinase |
| | | HGK |
| | | KIAA0687 |
| | | NIK |
| IGFBP3 | Insulin-like growth | IBP-3 |
| | factor-binding | IGF-binding protein 3 |
| | protein 3 | IGFBP-3 |
| | | IBP3 |

TABLE II
Panel of Tumor Autoantigens for CRC.

| Gene Symbol | Description | Synonyms |
|---|---|---|
| MAP4K4 | Mitogen-activated | HPK/GCK-like kinase HGK |
| | protein kinase | MAPK/ERK kinase kinase kinase 4 |
| | kinase kinase | MEK kinase kinase 4 |
| | kinase 4 | MEKKK 4 |
| | | Nck-interacting kinase |
| | | HGK |
| | | KIAA0687 |
| | | NIK |
| IGFBP3 | Insulin-like | IBP-3 |
| | growth factor- | IGF-binding protein 3 |
| | binding protein 3 | IGFBP-3 |
| | | IBP3 |
| TP53 | Cellular tumor | Antigen NY-CO-13 |
| | antigen p53 | Phosphoprotein p53 |
| | | Tumor suppressor p53 |
| | | p53 |
| IGF2BP2 | Insulin-like | IGF2 mRNA-binding protein 2 |
| | growth factor | IMP-2 |
| | 2 mRNA-binding | Hepatocellular carcinoma |
| | protein 2 | autoantigen p62 |
| | | IGF-II mRNA-binding protein 2 |
| | | VICKZ family member 2 |
| | | IMP2 |
| | | VICKZ2 |
| | | p62 |

TABLE III
Novel Tumor Autoantigens Used in Experimental Examples

| | | |
|---|---|---|
| NCBI | | |
| GenBank or | | |
| Protein | | |
| Accession | Description | Sequence |

Human Mitogen-Activated Kinase Kinase Kinase Kinase 4 (MAP4K4)
Protein. Note that the recombinant MAP4K4 which was used in
the Examples was amino acids 1-328 and contained an N-terminal
GST fusion tag (sequence not shown) commonly known to those
skilled in the art.

| | | |
|---|---|---|
| NP_004825 | Mitogen-activated | MANDSPAKSLVDIDLSSLRDPAGIFELVEVVGNGTYGQVYKGRHVKT |
| NP_060262 | protein kinase | GQLAAIKVMDVTEDEEEEIKLEINMLKKYSHHRNIATYYGAFIKKSP |
| NM_004834 | kinase kinase | PGHDDQLWLVMEFCGAGSITDLVKNTKGNTLKEDWIAYISREILRGL |
| | kinase 4 isoform | AHLHIHHVIHRDIKGQNVLLTENAEVKLVDFGVSAQLDRTVGRRNTF |
| | 1 (MAP4K4/HGK) | IGTPYWMAPEVIACDENPDATYDYRSDLWSCGITAIEMAEGAPPLCD |
| | SEQ ID NO: 1 | MHPMRALFLIPRNPPPRLKSKKWSKKFFSFIEGCLVKNYMQRPSTEQ |
| | | LLKHPFIRDQPNERQVRIQLKDHIDRTRKKRGEKDETEYEYSGSEEE |
| | | EEEVPEQEGEPSSIVNVPGESTLRRDFLRLQQENKERSEALRRQQLL |
| | | QEQQLREQEEYKRQLLAERQKRIEQQKEQRRRLEEQQRREREARRQQ |
| | | EREQRRREQEEKRRLEELERRRKEEEERRRAEEEKRRVEREQEYIRR |
| | | QLEEEQRHLEVLQQQLLQEQAMLLHDHRRPHPQHSQQPPPPQQERSK |
| | | PSFHAPEPKAHYEPADRAREVPVRTTSRSPVLSRRDSPLQGSGQQNS |
| | | QAGQRNSTSSIEPRLLWERVEKLVPRPGSGSSSGSSNSGSQPGSHPG |
| | | SQSGSGERFRVRSSSKSEGSPSQRLENAVKKPEDKKEVFRPLKPAGE |
| | | VDLTALAKELRAVEDVRPPHKVTDYSSSSEESGTTDEEDDDVEQEGA |
| | | DESTSGPEDTRAASSLNLSNGETESVKTMIVHDDVESEPAMTPSKEG |
| | | TLIVRQTQSASSTLQKHKSSSSFTPFIDPRLLQISPSSGTTVTSVVG |
| | | FSCDGMRPEAIRQDPTRKGSVVNVNPTNTRPQSDTPEIRKYKKRFNS |
| | | EILCAALWGVNLLVGTESGLMLLDRSGQGKVYPLINRRRFQQMDVLE |
| | | GLNVLVTISGKKDKLRVYYLSWLRNKILHNDPEVEKKQGWTTVGDLE |
| | | GCVHYKVVKYERIKFLVIALKSSVEVYAWAPKPYHKFMAFKSFGELV |
| | | HKPLLVDLTVEEGQRLKVIYGSCAGFHAVDVDSGSVYDIYLPTHVRK |
| | | NPHSMIQCSIKPHAIIILPNTDGMELLVCYEDEGVYVNTYGRITKDV |
| | | VLQWGEMPTSVAYIRSNQTMGWGEKAIEIRSVETGHLDGVFMHKRAQ |
| | | RLKFLCERNDKVFFASVRSGGSSQVYFMTLGRTSLLSW |

Human Insulin-Like Growth Factor Binding Protein 3 (IGFBP3/IBP3)
Protein. Note that the recombinant IGFBP3 which was used in the
Examples contained an N-terminal polyhistidine tag (sequence
not shown) commonly known to those skilled in the art.

| | | |
|---|---|---|
| NP_000589 | Insulin-like growth | MQRARPTLWAAALTLLVLLRGPPVARAGASSAGLGPVVRCEPCDARA |
| NM_000598 | factor-binding protein | LAQCAPPPAVCAELVREPGCGCCLTCALSEGQPCGIYTERCGSGLRC |
| | 3 isoform b precursor | QPSPDEARPLQALLDGRGLCVNASAVSRLRAYLLPAPPAPGNASESE |
| | (IGFBP3/IBP3) | EDRSAGSVESPSVSSTHRVSDPKFHPLHSKIIIIKKGHAKDSQRYKV |
| | (recombinant protein | DYESQSTDTQNFSSESKRETEYGPCRREMEDTLNHLKFLNVLSPRGV |
| | used was mature form, | HIPNCDKKGFYKKKQCRPSKGRKRGFCWCVDKYGQPLPGYTTKGKED |
| | amino acids 28-291) | VHCYSMQSK |
| | SEQ ID NO: 2 | |

1. A method of detecting antibodies related to colorectal cancer (CRC) in an individual comprising:
a. contacting a test sample from an individual with one or more target antigens of Table I; and
b. detecting binding of the one or more target antigens to one or more antibodies in the test sample, wherein the presence of the one or more antibodies bound against the one or more target antigens is indicative of colorectal cancer (CRC).

2. The method of claim 1, wherein the one or more target antigens are immobilized on a solid support.

3. The method of claim 1, wherein the test sample is contacted with all of the target antigens of Table I.

4. The method of claim 1, wherein the test sample is selected from the group consisting of cells, tissues or body fluids.

5. The method of claim 1, wherein the test sample is selected from the group consisting of blood, plasma or serum.

6. A method of detecting antibodies related to colorectal cancer (CRC) in an individual comprising:
a. contacting a test sample from the individual with at least two or more target antigens, each comprising an antigen of Table II, wherein at least one of said target antigens is selected from the group consisting of MAP4K4 and IGFBP3; and
b. detecting binding of the at least two or more target antigens to one or more antibodies in the test sample, wherein the presence of the one or more antibodies bound against the at least two or more target antigens is indicative of colorectal cancer (CRC).

7. The method of claim 6, wherein the at least two or more target antigens are immobilized on a solid support.

8. The method of claim 6, wherein the test sample is selected from the group consisting of cells, tissues or body fluids.

9. The method of claim 6, wherein the test sample is selected from the group consisting of blood, plasma or serum.

10. A method for identifying antibodies related to cancer, said method comprising:
a) comparing the gene expression level of one or more genes in cancer cells and normal cells; and
b) identifying one or more genes only activated in said cancer cells as compared to normal cells;
c) assaying body fluid from at least one individual with said cancer type for antibodies to the gene product of said genes identified in step b); and
d) identifying antibody reactive with at least one gene product assayed in step c).

11. The method of claim 10, wherein gene expression levels are determined by measuring mRNA.

12. The method of claim 10, wherein gene expression levels are determined by measuring protein.

13. The method of claim 10, wherein said normal cells are from normal tissues.

14. The method of claim 10, wherein said one or more genes identified in step b) are also not activated in non-recurrent cancer.

15. The method of claim 10, further comprising e) using the gene product reactive with said antibody of step c) to diagnose cancer in a person of unknown disease status.

16. A method for identifying antibodies related to cancer, said method comprising:
a) comparing the gene expression level of one or more genes in cancer cells and normal cells; and
b) identifying one or more genes activated more than 1.4 fold in said cancer cells as compared to normal cells;
c) assaying body fluid from at least one individual with said cancer type for antibodies to the gene product of said genes identified in step b); and
d) identifying antibody reactive with at least one gene product assayed in step c).

17. The method of claim 16, wherein gene expression levels are determined by measuring mRNA.

18. The method of claim 16, wherein gene expression levels are determined by measuring protein.

19. The method of claim 16, wherein said normal cells are from normal tissues.

20. The method of claim 16, wherein said body fluid is selected from the group consisting of serum and plasma.

21. The method of claim 16, further comprising e) using the gene product reactive with said antibody of step c) to diagnose cancer in a person of unknown disease status

22. The method of claim 16, wherein said one or more genes identified are activated more than 1.5 fold in said cancer cells as compared to normal cells.

23. The method of claim 16, wherein said one or more genes identified are activated more than 1.8 fold in said cancer cells as compared to normal cells.

24. The method of claim 16, wherein said one or more genes identified are activated more than 2.0 fold in said cancer cells as compared to normal cells.

25. The method of claim 16, wherein said one or more genes identified in step b) are also activated more than 1.4 fold in said cancer cells as compared to non-recurrent cancer.

26. The method of claim 16, wherein said cancer cells are from a solid tumor.


SEQUENCE LISTING (2 sequences)

SEQ ID NO: 1 - protein, 1166 aa, Homo sapiens
MANDSPAKSLVDIDLSSLRDPAGIFELVEVVGNGTYGQVYKGRHVKTGQLAAIKVMDVTEDEEEEIKLEINMLKKYSHHRNIATYYGAFIKKSPPGHDDQLWLVMEFCGAGSITDLVKNTKGNTLKEDWIAYISREILRGLAHLHIHHVIHRDIKGQNVLLTENAEVKLVDFGVSAQLDRTVGRRNTFIGTPYWMAPEVIACDENPDATYDYRSDLWSCGITAIEMAEGAPPLCDMHPMRALFLIPRNPPPRLKSKKWSKKFFSFIEGCLVKNYMQRPSTEQLLKHPFIRDQPNERQVRIQLKDHIDRTRKKRGEKDETEYEYSGSEEEEEEVPEQEGEPSSIVNVPGESTLRRDFLRLQQENKERSEALRRQQLLQEQQLREQEEYKRQLLAERQKRIEQQKEQRRRLEEQQRREREARRQQEREQRRREQEEKRRLEELERRRKEEEERRRAEEEKRRVEREQEYIRRQLEEEQRHLEVLQQQLLQEQAMLLHDHRRPHPQHSQQPPPPQQERSKPSFHAPEPKAHYEPADRAREVPVRTTSRSPVLSRRDSPLQGSGQQNSQAGQRNSTSSIEPRLLWERVEKLVPRPGSGSSSGSSNSGSQPGSHPGSQSGSGERFRVRSSSKSEGSPSQRLENAVKKPEDKKEVFRPLKPAGEVDLTALAKELRAVEDVRPPHKVTDYSSSSEESGTTDEEDDDVEQEGADESTSGPEDTRAASSLNLSNGETESVKTMIVHDDVESEPAMTPSKEGTLIVRQTQSASSTLQKHKSSSSFTPFIDPRLLQISPSSGTTVTSVVGFSCDGMRPEAIRQDPTRKGSVVNVNPTNTRPQSDTPEIRKYKKRFNSEILCAALWGVNLLVGTESGLMLLDRSGQGKVYPLINRRRFQQMDVLEGLNVLVTISGKKDKLRVYYLSWLRNKILHNDPEVEKKQGWTTVGDLEGCVHYKVVKYERIKFLVIALKSSVEVYAWAPKPYHKFMAFKSFGELVHKPLLVDLTVEEGQRLKVIYGSCAGFHAVDVDSGSVYDIYLPTHVRKNPHSMIQCSIKPHAIIILPNTDGMELLVCYEDEGVYVNTYGRITKDVVLQWGEMPTSVAYIRSNQTMGWGEKAIEIRSVETGHLDGVFMHKRAQRLKFLCERNDKVFFASVRSGGSSQVYFMTLGRTSLLSW

SEQ ID NO: 2 - protein, 291 aa, Homo sapiens
MQRARPTLWAAALTLLVLLRGPPVARAGASSAGLGPVVRCEPCDARALAQCAPPPAVCAELVREPGCGCCLTCALSEGQPCGIYTERCGSGLRCQPSPDEARPLQALLDGRGLCVNASAVSRLRAYLLPAPPAPGNASESEEDRSAGSVESPSVSSTHRVSDPKFHPLHSKIIIIKKGHAKDSQRYKVDYESQSTDTQNFSSESKRETEYGPCRREMEDTLNHLKFLNVLSPRGVHIPNCDKKGFYKKKQCRPSKGRKRGFCWCVDKYGQPLPGYTTKGKEDVHCYSMQSK